(19) United States
(12) Patent Application Publication
(10) Pub. No.: US 2007/0297999 A1
(43) Pub. Date: Dec. 27, 2007

(54) KIT CONTAINING AN ACIDIC COMPOSITION AND A COMPOSITION BASED ON A HYDROXYALKYL UREA

(75) Inventors: Angeles Fonolla Moreno, Paris (FR); Bertrand Plot, Paris (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/755,855

(22) Filed: May 31, 2007

Related U.S. Application Data
(60) Provisional application No. 60/817,069, filed on Jun. 29, 2006.

(30) Foreign Application Priority Data
Jun. 21, 2006 (FR) 06 52583

(51) Int. Cl. A61K 31/17; A61K 38/43; A61K 8/18; A61P 17/00

(52) U.S. Cl. 424/59; 424/94.1; 514/588

(57) ABSTRACT

The present invention relates to a kit containing: a composition A containing a physiologically acceptable acidic medium and at least one skincare and/or scalp-care agent; a composition B, packaged separately from composition A, composition B containing a physiologically acceptable basic medium and at least one hydroxyalkyl urea of formula (I) according to the invention.

### REFERENCE TO PRIOR APPLICATIONS

This application claims priority to U.S. provisional application 60/817,069 filed Jun. 29, 2006, and to French patent application 0652583 filed Jun. 21, 2006, both incorporated herein by reference.

### FIELD OF THE INVENTION

The present invention relates to a kit, preferably to a skincare and/or scalp-care kit, comprising:
a composition A comprising a physiologically acceptable acidic medium and at least one active agent such as a skincare and/or scalp-care agent;
a composition B, packaged separately from composition A, composition B comprising a physiologically acceptable basic medium and at least one hydroxyalkyl urea of formula (I) 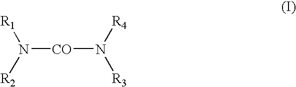
   in which R₁, R₂, R₃ and R₄ each independently represent a hydrogen atom, a C₁-C₄ alkyl group or a C₂-C₆ hydroxyalkyl group optionally comprising from 1 to 5 hydroxyl groups, at least one of the radicals R₁-R₄ representing a hydroxyalkyl group, and also the salts, solvates and isomers thereof.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Patent application EP 1 535 607 discloses the use of the hydroxyalkyl urea of formula (I) 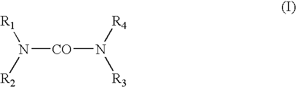
for its moisturizing properties in cosmetic compositions.

These compounds also give the composition containing them a light, non-greasy cosmetic feel, and make it easy to apply.

These compounds are especially advantageous in caring for dry skin and/or skin with areas that have become dehydrated and/or attacked following a treatment, such as a care treatment for greasy skin, an acne treatment or a depigmenting treatment especially comprising the application to the skin of active agents of acidic nature.

However, the inventors have observed that the stability of these compounds varies according to the pH. Many tests have in fact shown that these compounds are stable in basic media with a pH of greater than or equal to 7, but are unstable in acidic media with a pH of less than 6 and in particular less than 5. In these acidic media, the pH varies over time and, after two weeks, the Applicant finds an increase of more than one pH unit and a substantial decrease in viscosity at 35-45° C.

These variations in pH and viscosity thus necessitate a formulation of hydroxyalkyl ureas at a basic pH of greater than or equal to 7, which:
firstly, is incompatible with certain active agents that are stable, on the contrary, at acidic pH (e.g. depigmenting agents, greasy-skin care agents, slimming agents, etc.) or types of formulation;
and, secondly, requires a storage system that is reinforced with respect to microorganisms, especially via increasing the content of preserving agents, which may give rise, in particular in the case of individuals with sensitive and/or delicate and/or attacked skin, to sensations of skin discomfort, such as stinging, redness and heating.

### SUMMARY OF THE INVENTION

There is thus still a need for uses such as skincare and/or scalp care combining the hydroxyalkyl urea of formula (I) for its moisturizing properties and active agents such as skincare and/or scalp-care active agents of an acidic nature, while at the same time maintaining a good cosmetic nature for the compositions, i.e. good properties of stability and storage over time and also good efficacy and tolerance on the skin after application.

The inventors have discovered, unexpectedly, that a kit comprising:
a composition A containing a physiologically acceptable acidic medium and at least one active agent, for example at least one skincare and/or scalp-care agent;
a composition B, packaged separately from composition A, composition B containing a physiologically acceptable basic medium and at least one hydroxyalkyl urea of formula (I) 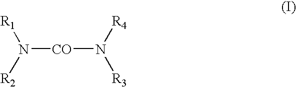
in which R₁, R₂, R₃ and R₄ each independently represent a hydrogen atom, a C₁-C₄ alkyl group or a C₂-C₆ hydroxyalkyl group optionally containing from 1 to 5 hydroxyl groups, at least one of the radicals R₁-R₄ representing a hydroxyalkyl group, and also the salts, solvates and isomers thereof,
   satisfies this need.

### BRIEF DESCRIPTION OF THE DRAWING

FIG. 1 shows a packaging device according to the invention.

### DETAILED DESCRIPTION OF THE PREFERRED EMBODIMENTS OF THE INVENTION

The kit according to the invention especially makes it possible to apply simultaneously or successively to the skin both care agents of an acidic nature and a hydroxyalkyl urea of formula (I) that is stable at basic pH, which would not be compatible in the same composition having a single pH.

Moreover, this kit makes it possible to apply to the skin a composition A and a composition B which, when mixed on the skin, give a pH for the mixture ranging from 4 to 6.5 and in particular a pH close to the physiological pH (pH 5.5), which thus respects the physiological state of the skin.

This kit is therefore suitable for caring for any type of skin and is advantageous for caring for sensitive skin, delicate skin (e.g.: baby skin) and/or attacked skin (e.g.: shaving, skin that has undergone an acidic treatment such as an acne treatment, a depigmenting treatment or a greasy-skin care treatment, etc.).

This kit may thus combine good efficacy of the care agents present in composition A with good moisturizing efficacy of the hydroxyalkyl urea present in composition B.

As another advantage, the kit according to the invention also makes it possible to reduce the total amount of preserving agents applied to the skin. Specifically, the amount of preserving agents for protecting composition A at acidic pH is minimal to nonexistent, which makes it possible to have, after applying composition A and composition B to the skin, a mixture that has a lower amount of preserving agents than a standard formulation containing hydroxyalkyl urea at pH 7 or higher. This reduction in the content of preserving agents is particularly desired, especially for caring for sensitive skin, delicate skin and/or attacked skin.

The invention thus preferably relates to a skincare and/or scalp-care kit comprising:
a composition A containing a physiologically acceptable acidic medium and at least one skincare and/or scalp-care agent;
a composition B, packaged separately from composition A, composition B containing a physiologically acceptable basic medium and at least one hydroxyalkyl urea of formula (I) 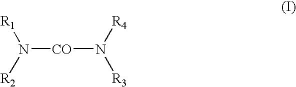
in which R₁, R₂, R₃ and R₄ each independently represent a hydrogen atom, a C₁-C₄ alkyl group or a C₂-C₆ hydroxyalkyl group optionally containing from 1 to 5 hydroxyl groups, at least one of the radicals R₁-R₄ representing a hydroxyalkyl group, and also the salts, solvates and isomers thereof.

The term “skincare and/or scalp-care agent” means an agent for enhancing the aesthetic appearance and/or comfort of the skin and/or the scalp. In particular, the agent may be intended for attenuating visible irregularities (dyschromia, hyperpigmentations, etc.) and/or tactile irregularities (wrinkles, subcutaneous fat, comedones, acne scars, etc.) of the skin and/or for making the skin healthier, less greasy and/or less dry.

According to one particular mode, the skincare agent present in composition A is other than a self-tanning agent or an enzyme.

According to another particular mode of the invention, the skincare agent is present in composition A in a content ranging from 0.0001% to 900% by weight relative to the total weight of the composition.

According to the invention, the term “physiologically acceptable medium” means a medium that is compatible with any keratin material such as the skin, including the scalp, the nails, mucous membranes, the eyes and the hair or any other area of bodily skin.

The medium according to the invention is preferably an aqueous medium, i.e. a medium comprising an amount of water of at least 50 by weight, preferably ranging from 30% to 99.4% by weight and better still from 30% to 95% by weight relative to the total weight of the composition.

According to the invention, the term “acidic medium” means a medium, especially an aqueous medium, with a pH of less than 6, in particular less than or equal to 5.5 and in practice chosen in the range from 1 to 5.5.

In particular, an acidic medium with a pH ranging from 1 to 5.5, preferably from 3 to 5.5 and even more preferentially from 4 to 5.5 will be used.

According to one particular mode, the kit according to the invention is characterized by an acidic medium with a pH of less than or equal to 5.

According to the invention, the term “basic medium” means a medium, especially an aqueous medium, with a pH of greater than or equal to 7.

Composition B at Basic pH

The hydroxyalkyl urea(s) present in composition B include at least one chosen from those corresponding to the general formula (I): 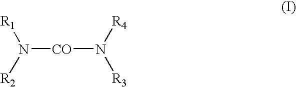
in which R₁, R₂, R₃ and R₄ each independently represent a hydrogen atom, a C₁-C₄ alkyl group or a C₂-C₆ hydroxyalkyl group optionally containing from 1 to 5 hydroxyl groups, at least one of the radicals R₁-R₄ representing a hydroxyalkyl group, and also the salts, solvates and isomers thereof.

In formula (I), mention may especially be made among the alkyl groups of methyl, ethyl, n-propyl, isopropyl, n-butyl, isobutyl and tert-butyl groups.

According to one particular mode of the invention, R₁ is a hydroxyalkyl group and R₂, R₃ and R₄ represent, independently of each other, a hydrogen atom or a C₁-C₄ alkyl group.

The preferred compounds of formula (I) are those containing only one hydroxyalkyl group, i.e. those for which R₁ is a hydroxyalkyl group and R₂, R₃ and R₄ represent, independently of each other, a hydrogen atom or a C₁-C₄ alkyl group.

The compounds of formula (I) for which R₁ is a hydroxyalkyl group and R₂, R₃ and R₄ each represent a hydrogen atom are more particularly preferred.

Among the hydroxyalkyl groups, those containing only one hydroxyl group and in particular hydroxyethyl, hydroxypropyl, hydroxybutyl, hydroxypentyl and hydroxyhexyl are preferred. The hydroxyethyl group is preferred.

According to one preferred mode, the compounds of formula (I) present in composition B are chosen from N-(2-hydroxyethyl)urea; N-(2-hydroxypropyl)urea; N-(3-hydroxypropyl)urea; N-(2,3-dihydroxypropyl)urea; N-(2,3, 4,5,6-pentahydroxyhexyl)urea; N-methyl-N-(1,3, 4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-[tris(hydroxymethyl)methyl]urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxy-ethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)-urea; N′,N′-bis(2-hydroxyethyl)-N′,N′-bis(2-hydroxy-propyl)urea, and mixtures thereof.

A compound that is particularly preferred for use in composition B is N-(2-hydroxyethyl)urea, which is referred to hereinbelow as “hydroxyethyl urea”.

The hydroxyalkyl ureas of formula (I) may be prepared as described in patent application DE-A-2 703 185. Among these, the hydroxyethyl urea is also commercially available, in the form of a mixture at 50% by weight in water, from the company National Starch under the trade name Hydrovance®.

Salts that may be mentioned include the salts of mineral acids, such as sulfuric acid, hydrochloric acid, hydrobromic acid, hydriodic acid, phosphoric acid and boric acid. Mention may also be made of the salts of organic acids, which may comprise one or more carboxylic acid, sulfonic acid or phosphonic acid groups. They may be linear, branched or cyclic aliphatic acids or alternatively aromatic acids. These acids may also comprise one or more hetero atoms chosen from O and N, for example in the form of hydroxyl groups. Mention may especially be made of propionic acid, acetic acid, terephthalic acid, citric acid and tartaric acid.

The term “solvate” means a stoichiometric mixture of the compound of formula (I) with one or more water molecules or with one or more organic solvent molecules. Such a mixture can be derived from the synthesis of the compound of formula (I). Useful solvate solvents include water, ethanol, methanol, etc.

The hydroxyalkyl urea(s) is (are) preferably present in composition B in a content that is effective for obtaining the desired moisturizing effect.

In particular, they will preferably be used in a content ranging for example from 0.01% to 50% by weight relative to the total weight of the composition, more preferentially from 0.1% to 20% by weight relative to the total weight of the composition and even more preferentially from 0.1% to 10% by weight relative to the total weight of the composition.

Composition A at Acidic pH

The skincare and/or scalp-care agent(s) present in composition A are preferably stable in an acidic medium as defined previously, i.e. their efficacy remains virtually constant at acidic pH.

On the other hand, in a medium at basic pH, the care agent becomes destabilized and/or loses its efficacy (biological) over time.

The preferred agents of acidic nature that are particularly suitable for use in the invention are generally organic acids, but other care agents may also be used, and a person skilled in the art will know how to select them as a function of their stability at acidic pH and the desired effect on the skin, in view of the present disclosure.

In particular, the care agent present in composition A may especially be chosen from an organic acid, a depigmenting agent, a desquamating agent, an antimicrobial agent, a calmative and/or anti-inflammatory agent, a sebo-regulating agent, a cicatrizing agent, an astringent and a slimming agent, and mixtures thereof.

According to a first embodiment, the care agent will be an organic acid. It will preferably be chosen from the group consisting of α-hydroxy acids, β-hydroxy acids, α-keto acids and β-keto acids, derivatives thereof and mixtures thereof.

In particular, the organic acid present in composition A may be chosen from the group consisting of ascorbic acid, kojic acid, caffeic acid, salicylic acid and derivatives thereof, citric acid, lactic acid, methyllacetic acid, glucuronic acid, glycolic acid, pyruvic acid, 2-hydroxybutanoic acid, 2-hydroxy-pentanoic acid, 2-hydroxyhexanoic acid, 2-hydroxy-heptanoic acid, 2-hydroxyoctanoic acid, 2-hydroxy-nonanoic acid, 2-hydroxydecanoic acid, 2-hydroxy-undecanoic acid, 2-hydroxydodecanoic acid, 2-hydroxy-tetradecanoic acid, 2-hydroxyhexadecanoic acid, 2-hydroxyoctadecanoic acid, 2-hydroxytetracosanoic acid, 2-hydroxyeicosanoic acid, mandelic acid, benzoic acid, phenylacetic acid, gluconic acid, galacturonic acid, aleuritic acid, ribonic acid, tartronic acid, tartaric acid, malic acid, fumaric acid, retinoic acid and derivatives thereof, benzene-1,4-bis(3-methylidene-10-camphorsulfonic acid), urocanic acid, 2-phenylbenzimidazole-5-sulfonic acid, α-(2-oxo-3-bornylidene)-4-toluenesulfonic acid, 2-hydroxy-4-methoxy-5-sulfonic acid, plant extracts containing acids and more especially fruit extracts, xanthic acid derivatives, β-glycyrrhetinic acid and asiatic acid, and mixtures thereof.

Other skincare and/or scalp-care agents are also suitable for use in the invention. Examples of agents that may be used in composition A at acidic pH are listed below and classified as a function of the desired type of care on the skin, it being understood that a person skilled in the art can combine them in the same care to obtain complementary effects aimed at enhancing the appearance and/or comfort of the skin.

The component according to the invention may especially constitute care for greasy skin, care for aged skin, care for dry skin, care aimed at depigmenting the skin or attenuating hyperpigmented areas and/or lesions of the skin (e.g.: acne scars), slimming care for the body, care for sensitive skin, for delicate skin (e.g.: baby skin) or for attacked skin (e.g.: shaving, etc.), bodily or facial scrubbing care, etc.

Depigmenting Care

As depigmenting agents that may be used in composition A according to the invention, mention may be made especially of ascorbic acid (vitamin C) and derivatives thereof and especially vitamins CG, CP and 3-O ethyl vitamin C, kojic acid, tranexamic diacid and derivatives thereof, gentisic acid and derivatives thereof, dioic acid, lipoic acid, ellagic acid, and linoleic acid and derivatives thereof.

Ascorbic acid and derivatives thereof or kojic acid and derivatives thereof will preferably be used as depigmenting agent.

Greasy-Skin Care

Useful greasy-skin care agents include desquamating agents, antimicrobial agents, calmatives, anti-inflammatory agents, sebo-regulating agents and antioxidants, and mixtures thereof.

Preferably, composition A according to the invention will comprise as care agent a sebo-regulating agent, i.e. an agent capable of regulating the activity of the sebaceous glands.

According to one particular mode, composition A may also optionally comprise an antimicrobial agent.

More preferably, it may also comprise a desquamating agent.

Moreover, to further increase the efficacy and/or tolerance of the composition A, calmatives or anti-inflammatory agents, antioxidants, cicatrizing agents and astringents, and mixtures thereof, may also be added.

As sebo-regulating agents that may be used in composition A according to the invention, mention may be made especially of:
retinoic acid and derivatives thereof;
zinc salts such as zinc gluconate, zinc pyrrolidonecarboxylate (or zinc pidolate), zinc lactate, zinc aspartate, zinc carboxylate, zinc salicylate or zinc cysteate;
copper salts, in particular copper pidolate;
sebacic acid, sold especially in the form of a sodium polyacrylate gel under the name Sebosoft by the company Sederma;
glycine grafted onto an undecylenic chain, such as the product sold under the name Lipacide UG OR by the company SEPPIC;
the mixture of oleanolic acid and of nordihydroguaiaretic acid, such as the product sold in the form of a gel under the name AC.Net by the company Sederma;
phthalimidoperoxyhexanoic acid;
10-hydroxydecanoic acid, and especially mixtures of 10-hydroxydecanoic acid, of sebacic acid and of 1,10-decanediol, such as the product sold under the name Acnacidol® BG by the company Vincience;
aluminium salts, among which mention may be made of aluminium chlorohydrate, aluminium chlorohydrex, aluminium chlorohydrex PEG, aluminium chlorohydrex PG, aluminium dichlorohydrate, aluminium dichlorohydrex PEG, aluminium dichlorohydrex PG, aluminium sesquichlorohydrate, aluminium sesquichlorohydrex PEG, aluminium sesquichlorohydrex PG, alum salts, aluminium sulfate, aluminium zirconium octachlorohydrate, aluminium zirconium pentachlorohydrate, aluminium zirconium tetrachlorohydrate, aluminium zirconium trichlorohydrate, and more particularly the aluminium chlorohydrate sold by the company Reheis under the name Microdry Aluminum Chlorohydrate or by the company Guilini Chemie under the name Aloxicoll PF 40. Examples of aluminium and zirconium salts include the salt sold by the company Reheis under the name Reach AZP-908-SUF, and “activated” aluminium salts, for example the product sold by the company Reheis under the name Reach 103 or by the company Westwood under the name Westchlor 200.

According to the invention, the term “antimicrobial agents” means agents that have effects on the specific flora of greasy skin, for instance P. acnes. These effects either may be bactericidal or anti-bacterial adhesion (preventing and/or reducing the adhesion of microorganisms), or may act on the biofilm of bacteria to prevent their multiplication.

As antimicrobial agents that may be used in composition A according to the invention, included are citrollic acid, sperillic acid, 10-hydroxy-2-decanoic acid, for instance Acnacidol P from Vincience, azelaic acid, Arlatone dioic acid from Uniqema, phthalimidoperoxyhexanoic acid or Eureco HC from Chemron Corporation; ellagic acid; undecylenic acid and salts thereof, 3-hydroxybenzoic acid, 4-hydroxybenzoic acid, phytic acid, lipoic acid, azelaic acid and salts thereof, arachidonic acid, octanoylglycine (Lipacide C8G® from SEPPIC), and salicylic acid and derivatives thereof.

The term “desquamating agent” means any compound capable of acting either directly on desquamation by promoting exfoliation, or on the enzymes involved in the desquamation or degradation of corneodesmosomes, glycosidases, stratum corneum chymotryptic enzyme (SCCE), or even other proteases (trypsin, chymotrypsin-like).

As desquamating agents that may be used in composition A of the invention, included are:
β-hydroxy acids, in particular salicylic acid and derivatives thereof (including 5-n-octanoylsalicylic acid); α-hydroxy acids, such as glycolic acid, citric acid, lactic acid, tartaric acid, malic acid or mandelic acid; gentisic acid and derivatives thereof; cinnamic acid; jasmonic acid and derivatives thereof, as in patent applications EP 1 333 022 and EP 1 333 021;
(N-2-hydroxyethylpiperazin-N-2-ethane)sulfonic acid (HEPES); 2-oxathiazolidine-4-carboxylic acid (procysteine); α-amino acid derivatives of glycine type (as described in EP 0 852 949, and also the sodium methyl glycine diacetate sold by BASF under the trade name Trilon M); (3-hydroxy-2-pentylcyclopentyl)acetic acid.

Preferred desquamating agents that may be mentioned include β-hydroxy acids, such as 5-n-octanoylsalicylic acid; glycolic acid, citric acid, lactic acid, tartaric acid, malic acid or mandelic acid; (N-2-hydroxyethyl-piperazine-N-2-ethane)sulfonic acid (HEPES); and mixtures thereof.

As particular calmatives that may be used in composition A according to the invention, included are: caffeine and derivatives thereof, β-glycyrrhetinic acid and salts or derivatives thereof (stearyl glycyrrhetate, 3-stearoyloxyglycyrrhetic acid, glycyrrhetinic acid monoglucuronide) and also plants containing them (e.g.: Glycyrrhiza glabra), oleanolic acid and salts thereof, ursolic acid and salts thereof, boswellic acid and salts thereof, betulinic acid and salts thereof; an extract of mint, in particular of mint leaves, for instance Calmiskin® from Silab, an extract of rose, for instance Herbasol rose extract, or Stimu-tex AS from Pentapharm.

A mint extract, in particular from mint leaves, such as Calmiskin® from Silab, a rose extract, such as Herbasol rose extract, Stimu-tex AS from Pentapharm, or β-glycyrrhetinic acid and salts or derivatives thereof, will preferably be used.

As particular anti-inflammatory agents that may be used in composition A according to the invention, mention may be made especially of azelaic acid.

As cicatrizing agents that may be used in composition A according to the invention, mention may be made especially of certain amino acids, for instance hydroxyproline, arginine or serine; allantoin; ximeninic acid and salts thereof such as ximeninic acid from Indena, and Herbasol lemon from Cosmetochem.

The preferred cicatrizing agents are hydroxyproline, arginine, serine and allantoin.

As astringents (agents for combating dilation of the sebaceous follicles) that may be used in composition A according to the invention, mention may be made especially of aluminium salts, alum salts and extract of Hamamelis, and mixtures thereof.

The alums that may be used are especially chosen from:
the potassium alum of structure: KAl(SO₄)₂.12H₂O
the ammonium alum of structure: NH₄Al(SO₄)₂.12H₂O
the sodium alum of structure: NaAl(SO₄)₂.12H₂O
aluminium sulfate salts of structure: Al₂(SO₄)₃.nH₂O in which n is from 0 to 27.

The alums may be in the form of powder or crystals or in dissolved form. The potassium alum will be used more particularly, preferably in a content ranging from 0.1% to 3% by weight relative to the total weight of composition A and preferentially in a content ranging from 1% to 2% by weight relative to the total weight of the composition.

The preferred astringents are alums and extract of Hamamelis.

Slimming Care

As slimming agents that may be used in composition A according to the invention, mention may be made especially of caffeine, nicotinic acid and derivatives thereof, more particularly such as α-tocopheryl nicotinate and hexyl nicotinate (see especially in this respect document EP-A-371 844).

As preferred care agent present in composition A, mention may be made especially of ascorbic acid and derivatives thereof, kojic acid, zinc salts, copper salts, 5-n-octanoylsalicylic acid, (N-2-hydroxy-ethylpiperazine-N-2-ethane)sulfonic acid, glycolic acid, citric acid, lactic acid, tartaric acid, malic acid or mandelic acid, alum salts, an extract of Hamamelis, an extract of rose, an extract of mint, β-glycyrrhetinic acid, allantoin, serine, arginine, hydroxyproline, caffeine or nicotinic acid and derivatives thereof.

The skincare and/or scalp-care agent will generally be present in composition A according to the invention in an amount that is effective for the desired effect, in particular the desired depigmenting effect, greasy-skin-combating effect and/or slimming effect.

By way of non-limiting example, the skincare and/or scalp-care agent will be present in composition A in a content ranging from 0.001% to 90% by weight relative to the total weight of composition A, in particular from 0.01% to 30% by weight relative to the total weight of composition A and even more preferentially from 0.05% to 10% or even from 0.1% to 1% by weight relative to the total weight of composition A.

Composition A may also comprise cosmetic adjuvants such as those described in the galenical section that follows. In particular, it may comprise gelling agents that are stable at acidic pH and follicle-targeting agents suitable for formulation in acidic medium.

These follicle-targeting agents are particularly advantageous in caring for greasy skin.

Hydrophilic or lipophilic gelling agents that may especially be mentioned include Carbopols, Luvigels, Hostacerin AMPS, Simulgel, Sepigels, xanthan gum, guar gum, cellulose gum and alginates, and mixtures thereof. Hectorites may also be mentioned.

Follicle-targeting agents that may be mentioned include constituent polymers porous particles. These particles preferably have a mean volume diameter of less than or equal to 10 μm. Specifically, such particles can enter the sebaceous follicles by applying a mechanical force.

This mechanical force generally originates from massaging, which, besides the thrust it exerts, generates a pump effect on the follicles. The particles gradually reach the follicular canal in which they are able to absorb sebum and, where appropriate, to release the active compound they contain. The constituent material of the particles is then discharged by virtue of the flow of sebum and/or the growth of the hair, thus making it possible to avoid any possible adverse reaction of the body to this material.

In particular, porous particles, especially spherical particles, with a mean numerical diameter possibly ranging from 0.1 to 50 μm, especially from 0.1 to 20 μm and most particularly from 0.5 to 10 μm will be used.

Preferred porous particles that may be used include polyamide particles, in particular of nylon 6, nylon 6-6, nylon 12 or nylon 6-12 such as those sold by the company Atofina under the generic name Orgasol.

This encapsulation system that allows targeting of the follicles is particularly advantageous in compositions intended for treating greasy skin.

According to one particular mode of the invention, to reinforce and/or complement the effect of the care agent present in composition A at acidic pH according to the invention, composition B at basic pH according to the invention may also comprise at least one additional care agent known to be stable at basic pH.

According to the invention, the expression “stable at basic pH” refers to an agent that has, at pH 7, efficacy (biological) that is virtually constant over time. On the other hand, in a very acidic medium, this active agent loses its efficacy.

A person skilled in the art will know how to select, from lists of additional cosmetic and/or dermatological care agents, those that would be suitable for composition B at basic pH according to the invention.

These additional agents may be chosen especially from depigmenting agents, antimicrobial agents, antiperspirants, metal-chelating agents, hydrolysed proteins, antioxidants, vitamins, anti-inflammatory agents, anti-irritant agents or calmatives, moisturizers, plant extracts, agents for improving the barrier function, matting agents, abrasive fillers or exfoliants, desquamating agents, sebo-regulating agents, cicatrizing agents, astringents, fillers, optical brighteners, fluorescers, anti-ageing agents and slimming agents, and mixtures thereof.

Humectants or moisturizers that may especially be mentioned include glycerol and derivatives thereof, xylitol, chitosan and derivatives thereof, collagen, plankton, and an extract of Imperata cylindra sold under the name Moist 24® by the company Sederma.

Agents for improving the barrier function that may especially be mentioned include ceramides and derivatives, sphenoid-based compounds, glycosphingo-lipids, phospholipids, cholesterol and derivatives thereof, phytosterols, essential fatty acids, diacylglycerol, 4-chromanone and chromone derivatives, petroleum jelly, lanolin, shea butters, cocoa butter and lanolin.

Depigmenting agents that may especially be mentioned include α- and β-arbutin, lucinol and derivatives thereof, resorcinol and derivatives thereof, calcium D-pantheteine sulfonate, vitamin B3, ceramides and their homologues, plant derivatives such as chamomile, bearberry, the aloe family (A. vera, ferox or bardensis), mulberry or skullcap, without this list being exhaustive.

Matting agents that may especially be mentioned include a rice starch or a corn starch, kaolinite, silicas, talc, an extract of pumpkin seeds, cellulose microbeads, plant fibre, synthetic fibres, in particular polyamides, expanded acrylic copolymer microspheres, polyamide powders, silica powders, polytetrafluoroethylene powders, silicone resin powders, acrylic copolymer powders, wax powders, polyethylene powders, powders of elastomeric crosslinked organopolysiloxane coated with silicone resin, talc/titanium dioxide/alumina/silica composite powders, amorphous mixed silicate powders, acrylic polymer powders, and silicate particles, especially mixed silicate particles, and mixtures thereof.

Sebo-regulating or anti-seborrhoeic agents that may be mentioned include:
mixtures of extract of cinnamon, of tea and octanoylglycine, such as Sepicontrol A5 TEA from SEPPIC;
the mixture of capryloyl glycine, of sarcosine and of extract of Cinnamomum zeylanicum sold especially by the company SEPPIC under the trade name Sepicontrol A5®;
extracts of plants of the species Arnica montana, Cinchona succirubra, Eugenia carophyllata, Humulus lupulus, Hypericum perforatum, Mentha piperita, Rosmarinus officinalis, Salvia officinalis and Thymus vulgaris, all sold, for example, by the company Maruzen;
extracts of European meadowsweet (Spiraea ulmaria), such as the product sold under the name Sebonormine® by the company Silab;
extracts of the alga Laminaria saccharina, such as the product sold under the name Phlorogine® by the company Biotechmarine;
extracts of sugar cane, such as the product sold under the name Policasonol® by the company Sabinsa;
mixtures of extracts of roots of salad burnet (Sanguisorba officinalis/Poterium officinale), of rhizomes of ginger (Zingiber officinalis) and of bark of cinnamon tree (Cinnamomum cassia), such as the product sold under the name Sebustop® by the company Solabia;
extracts of linseed, such as the product sold under the name Linumine® by the company Lucas Meyer;
extracts of Phellodendron, such as those sold under the name Phellodendron extract BG by the company Maruzen or Oubaku liquid B by the company Ichimaru Pharcos;
mixtures of argan oil, of extract of Serenoa serrulata (saw palmetto) and of extract of sesame seed, such as the product sold under the name Regu SEB® by the company Pentapharm;
mixtures of extracts of willowherb, of Terminalia chebula, of nasturtium and of bioavailable zinc (microalgae), such as the product sold under the name Seborilys® by the company Green Tech;
extracts of Pygeum afrianum, such as the product sold under the name Pygeum afrianum sterolic lipid extract by the company Euromed;
extracts of Serenoa serrulata, such as the products sold under the name Viapure Sabal by the company Actives International or those sold by the company Euromed;
mixtures of extracts of plantain, of Berberis aquifolium and of sodium salicylate, such as the product sold under the name Seboclear® by the company Rahn;
extract of clove, such as the product sold under the name Clove extract powder by the company Maruzen;
argan oil, such as the product sold under the name Lipofructyl® by Laboratoires Sérobiologiques;
lactic protein filtrates, such as the product sold under the name Normaseb® by the company Sederma;
extracts of the alga laminaria, such as the product sold under the name Laminarghane® by the company Biotechmarine;
oligosaccharides from the alga Laminaria digitata, such as the product sold under the name Phycosaccharide AC by the company Codif;
sulfonated shale oil, such as the product sold under the name Ichthyol Pale by the company Ichthyol;
extracts of meadowsweet (Spiraea ulmaria), such as the product sold under the name Cytobiol Ulmaire by the company Libiol;
glucomannans extracted from konjac tuber and modified with alkylsulfonate chains, such as the product sold under the name Biopol Beta by the company Arch Chemical;
extracts of Sophora angustifolia, such as those sold under the name Sophora powder or Sophora extract by the company Bioland;
extracts of Cinchona succirubra bark, such as the product sold under the name Red Bark HS by the company Alban Muller;
extracts of Quillaja saponaria, such as the product sold under the name Panama wood HS by the company Alban Muller;
tri(C₁₂-C₁₃)alkyl citrate sold under the name Cosmacol® ECI by the company Sasol; tri(C₁₄-C₁₅)alkyl citrate sold under the name Cosmacol® ECL by the company Sasol;
specific PPAR-γ activators, for instance those described in patent application WO 2005/053 632;
extracts of plants of the genus Silybum;
sapogenins or plant extracts containing from, in particular extracts of diosgenin-rich Dioscorea plants;
samphire;
extracts of Eugenia carophyllata containing eugenol and eugenyl glucoside, and mixtures thereof.

Desquamating agents that may especially be mentioned include oligofucoses, extract of Sophora japonica; resveratrol, honey; sugar derivatives such as O-octanoyl-6-D-maltose and N-acetylglucosamine.

Calmatives or anti-irritant agents that may especially be mentioned include vitamins E, B5 and B3, dextran sulfate, an extract of Paeonia suffruticosa and/or lactiflora, phycosaccharides from the company Codif, an extract of Laminaria saccharina, extracts of Centella asiatica, canola oil, extracts of camomile, allantoin, omega-3-unsaturated oils such as musk rose oil, blackcurrant oil, Ecchium oil or fish oil, beauty-leaf oil, extracts of plankton, capryloyl glycine, Seppicalm VG® (Nymphea alba and sodium palmitoylproline) from SEPPIC, an extract of Pygeum, an extract of Boswellia serrata, an extract of Centipeda cunnighami, an extract of Helianthus annus, in particular Helioxine from Silab, an extract of Linum usitatissimum, for instance Sensiline from Silab, tocotrienols, extracts of Cola nitida, piperonal, an extract of clove, an extract of Epilobium angustifolium, Aloe vera, an extract of Bacopa moniera, phytosterols, cornflower water, rosewater, dextran, as in Modulene® from Vincience, an extract of mint, in particular of mint leaves, for instance Calmiskin® from Silab, aniseed derivatives, filamentous bacteria, for instance Vitreoscillal filiformis as described in patent EP 761 204, an extract of rose, for instance Herbasol rose extract, Stimu-tex AS from Pentapharm, alkaline-earth metal salts, especially of strontium, and niacinamide, and mixtures thereof.

Antioxidants that may especially be mentioned include tocopherol and esters thereof, in particular tocopheryl acetate; BHT and BHA.

Mention may also be made of polyphenols, epigallocatechins and natural extracts containing them, anthocyans, extracts of rosemary, extracts of olive leaves, green tea, resveratrol and derivatives thereof, pycnogenol, ergothineine, N-acetylcysteine, biotin, chelating agents, idebenone, plant extracts, for instance Pronalen Bioprotect™ from the company Provital, free-radical scavengers, for instance vitamin E, coenzyme Q10, bioflavonoids, SODs, phytanetriol, lignans, melatonin, pidolates and glutathione.

Astringents that may be mentioned include extracts of skullcap, extracts of meadowsweet, extracts of European meadowsweet, extracts of gentian and extracts of burdock, and mixtures thereof.

Anti-ageing agents that may especially be mentioned include:
among the active agents that stimulate epidermal macromolecules, such as fillagrin and keratins, mention may be made especially of the extract of lupin sold by the company Silab under the trade name Structurine®; the extract of buds of the beech tree Fagus sylvatica sold by the company Gattefosse under the trade name Gatuline® RC; and the extract of the zooplankton Salina sold by the company Seporga under the trade name GP4G®; copper tripeptide from Procyte; a peptide extract of Voandzeia substerranea, such as the product sold by the company Laboratoires Sérobiologiques under the trade name Filladyn LS 9397®;
among the dermo-decontracting agents, mention may be made especially of manganese gluconate and other salts, alverine citrate and salts thereof, glycine, an extract of Iris pallida, a hexapeptide (Argeriline R from Lipotec) or sapogenins, for instance wild yam and the carbonyl amines described in patent application EP 1 484 052.

Slimming (lipolytic) agents that may especially be mentioned include theophylline and its derivatives, theobromine, acefylline, aminophylline, chloroethyl-theophylline, diprofylline, diniprophylline, etamiphylline and its derivatives, etofylline and proxyphylline; extracts of tea, of coffee, of guarana, of maté, of cola (Cola nitida) and especially the dry extract of guarana fruit (Paulina sorbilis) containing 8% to 10% caffeine; extracts of climbing ivy (Hedera helix), of arnica (Arnica montana L), of rosemary (Rosmarinus officinalis N), of marigold (Calendula officinalis), of sage (Salvia officinalis L), of ginseng (Panax ginseng), of St.-John's wort (Hypericum perforatum), of butcher's-broom (Ruscus aculeatus L), of meadowsweet (Filipendula ulmaria L), of orthosiphon (Orthosiphon stamincus Benth), of birch (Betula alba), of pumpwood and of argan tree, extracts of ginkgo biloba, extracts of horsetail, extracts of escin, extracts of cangzhu, extracts of Chrysanthellum indicum, extracts of diosgenin-rich Dioscorea plants or pure diosgenin or hecogenin and derivatives thereof, extracts of Ballota, extracts of Guioa, of Davallia, of Terminalia, of Barringtonia, of Trema or of Antirobia.

The additional active agent(s) and/or ingredients used in composition B according to the invention may represent from 0.0001% to 20%, preferably from 0.01 to 10% and better still from 0.01% to 1% by weight relative to the total weight of the composition.

A person skilled in the art will know how to select from the preceding lists the additional agents that are particularly suitable for the basic medium of the composition B according to the invention and that do not affect the properties of the hydroxyalkyl urea.

The compositions according to the invention are generally preferably suitable for topical application to the skin and generally comprise a physiologically acceptable medium, i.e. a medium that is compatible with the skin. This is a cosmetically acceptable medium, i.e. a medium that has a pleasant colour, odour and feel and that does not generate unacceptable discomfort (stinging, tautness or redness) liable to put the consumer off using this composition.

The compositions of the present invention may be in any form, especially including a form that is suitable for topical application, especially in the form of aqueous gels, in the form of emulsions obtained by dispersing a fatty phase (also known as an oily phase) in an aqueous phase (O/W) or, conversely, (W/O) or multiple emulsions (for example W/O/W, O/W/o or O/O/W). They may be more or less fluid and may have the appearance of a white or coloured cream, a pomade, a milk, a lotion, a serum, a paste, a powder or a solid tube, and they may optionally be packaged as an aerosol and be in the form of a mousse or spray. These compositions are prepared according to the usual methods.

According to one particular embodiment of the invention, the compositions of the invention may be in the form of an emulsion and in this case may comprise at least one oily phase. The proportion of the oily phase of the emulsion may preferably range from 1% to 80% by weight, more preferably from 2% to 50% by weight and better still from 2% to 40% by weight relative to the total weight of the composition. The fatty substances of the oily phase, especially the oils, and the emulsifiers and coemulsifiers that may be present, used in the composition in emulsion form are chosen from those conventionally used in cosmetics or dermatology. The emulsifier and the coemulsifier, when they are present, are generally in a proportion ranging from 0.1% to 30% by weight, preferably from 0.3% to 20% by weight and better still from 0.5% to 15% by weight relative to the total weight of the composition. The emulsion may also contain lipid vesicles in addition to or instead of the emulsifiers and/or coemulsifiers.

The emulsions generally contain at least one emulsifier chosen from amphoteric, anionic, cationic and nonionic emulsifiers, used alone or as a mixture. The emulsifiers are chosen in an appropriate manner according to the continuous phase of the emulsion to be obtained (W/O or O/W). When the emulsion is multiple, it generally comprises an emulsifier in the primary emulsion and an emulsifier in the outer phase into which the primary emulsion is introduced.

As emulsifiers that may be used for the preparation of the W/O emulsions, examples that may be mentioned include alkyl esters or ethers of sorbitan, of glycerol or of sugars; silicone surfactants, for instance dimethicone copolyols such as the mixture of cyclomethicone and of dimethicone copolyol, sold under the names DC 5225 C and DC 3225 C by the company Dow Corning, and alkyldimethicone copolyols such as laurylmethicone copolyol sold under the name “Dow Corning 5200 Formulation Aid” by the company Dow Corning, cetyl dimethicone copolyol sold under the name Abil EM 90® by the company Goldschmidt, and the mixture of Polyglyceryl-4 isostearate/cetyl dimethicone copolyol/hexyl laurate sold under the name Abil WE 09® by the company Goldschmidt. One or more co-emulsifiers may also be added thereto, which may be chosen, advantageously, from the group comprising branched-chain fatty acid esters of polyol, and especially branched-chain fatty acid esters of glycerol and/or of sorbitan, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

As emulsifiers that may be used for the preparation of the O/W emulsions, examples that may be mentioned include nonionic emulsifiers such as fatty acid esters of oxyalkylenated (more particularly polyoxyethylenated) polyols, for example polyethylene glycol stearates, for instance PEG-100 stearate, PEG-50 stearate and PEG-40 stearate; fatty acid esters of oxyalkylenated sorbitan comprising, for example, from 20 to 100 EO, for example those sold under the trade names Tween 20 or Tween 60 by the company Uniqema; oxyalkylenated (oxyethylenated and/or oxypropylenated) fatty alkyl ethers; alkoxylated or non-alkoxylated sugar esters, for instance sucrose stearate such as PEG-20 methylglucose sesquistearate; sorbitan esters such as the sorbitan palmitate sold under the name Span 40 by the company Uniqema; diacid esters of fatty alcohols, for instance dimyristyl tartrate; mixtures of these emulsifiers, for instance the mixture of glyceryl stearate and of PEG-100 stearate (CTFA name: Glyceryl Stearate/PEG-100 Stearate) sold under the name Arlacel 165 by the company Uniqema and under the name Simulsol 165 by the company SEPPIC; or the mixture of dimyristyl tartrate, cetearyl alcohol, Pareth-7 and PEG-25 laureth-25, sold under the name Cosmacol PSE by the company Sasol (CTFA name: Dimyristyl tartrate/cetearyl alcohol/12-15 Pareth 7/PPG 25 laureth 25).

Coemulsifiers such as, for example, fatty alcohols containing from 8 to 26 carbon atoms, for instance cetyl alcohol, stearyl alcohol and the mixture thereof (cetearyl alcohol), octyldodecanol, 2-butyloctanol, 2-hexyldecanol, 2-undecylpentadecanol or oleyl alcohol, or fatty acids, may be added to these emulsifiers.

It is also possible to prepare emulsions without emulsifying surfactants or containing less than 0.5% of them relative to the total weight of composition (A) or (B), by using suitable compounds, for stabilizing the emulsions, for example amphiphilic polymers or electrolytes.

When the composition of the invention is in emulsion form, it comprises at least one oily phase that contains at least one oil, especially a cosmetic oil. The term “oil” means a fatty substance that is liquid at room temperature (25° C.).

As oils that may be used in the composition of the invention, it is possible to use, for example, hydrocarbon-based oils of animal origin, such as perhydrosqualene (or squalane); hydrocarbon-based oils of plant origin, such as caprylic/capric acid triglycerides, for instance those sold by the company Stearineries Dubois or those sold under the names Miglyol 810, 812 and 818 by the company Dynamit Nobel, or alternatively oils of plant origin, for instance sunflower oil, corn oil, soybean oil, marrow oil, grapeseed oil, sesame seed oil, hazelnut oil, apricot oil, macadamia oil, arara oil, coriander oil, castor oil, avocado oil, jojoba oil and shea butter oil; synthetic oils; silicone oils, for instance volatile or non-volatile polymethylsiloxanes (PDMS) containing a linear or cyclic silicone chain, which are liquid or pasty at room temperature; fluoro oils, such as partially hydrocarbon-based and/or silicone-based fluoro oils, for instance those described in document JP-A-2 295 912; ethers, such as dicaprylyl ether (CTFA name: Dicaprylyl ether); C₁₂-C₁₅ fatty alkyl benzoates (Finsolv TN from Finetex); arylalkyl benzoate derivatives, for instance 2-phenylethyl benzoate (X-Tend 226 from ISP); and amido oils, for instance isopropyl lauroylsarcosinate (Eldew SL-205 from Ajinomoto); and mixtures thereof.

The compositions of the invention may also contain one or more organic solvents that may be chosen from the group consisting of hydrophilic organic solvents, lipophilic organic solvents and amphiphilic solvents, or mixtures thereof.

Examples of hydrophilic organic solvents that may be mentioned include linear or branched monohydric alcohols containing from 1 to 8 carbon atoms, for instance ethanol, propanol, butanol or isobutanol; polyethylene glycols containing from 6 to 80 ethylene oxides; polyols such as propylene glycol, isoprene glycol, butylene glycol, glycerol or sorbitol; monoalkyl or dialkyl isosorbides in which the alkyl groups containing from 1 to 5 carbon atoms, for instance dimethyl isosorbide; glycol ethers, for instance diethylene glycol monomethyl ether or monoethyl ether and propylene glycol ethers, for instance dipropylene glycol methyl ether.

Amphiphilic organic solvents that may be mentioned include polypropylene glycol (PPG) derivatives such as fatty acid esters of polypropylene glycol, and derivatives of PPG and of fatty alcohols, for instance PPG-23 oleyl ether, and PPG-36 oleate.

Examples of lipophilic organic solvents that may be mentioned include fatty esters such as diisopropyl adipate, dioctyl adipate or alkyl benzoates.

The compositions in accordance with the present invention may also comprise cosmetic adjuvants chosen for example from softeners, humectants, opacifiers, stabilizers, emollients, silicones, antifoams, fragrances, preserving agents, anionic, cationic, nonionic, zwitterionic or amphoteric surfactants, fillers, polymers, propellants, and acidifying or basifying agents, or any other ingredient usually used in cosmetics and/or dermatology.

Hydrophilic thickeners that may be mentioned include carboxyvinyl polymers such as carbopols (carbomers) and the Pemulen products (acrylate/C10-C30-alkylacrylate copolymer); cellulose derivatives such as hydroxyethylcellulose; polysaccharides and especially gums such as xanthan gum; and mixtures thereof.

Lipophilic thickeners that may be mentioned include modified clays such as hectorite and derivatives thereof, for instance the products sold under the name Bentone.

A person skilled in the art will know how to adapt the choice of cosmetic adjuvants and the contents thereof as a function of the pH (acidic or basic) of the compositions according to the invention.

Cosmetic Kit

Compositions A and B used for the implementation of the invention are packaged separately in separate, e.g., two, compartments, formed either of two separate containers, or in a single device.

The term “single device” means a device via which the two compartments are solidly attached. Such a device may be obtained via a process of moulding the two compartments as a single article, especially made of a thermoplastic material. It may also result from any form of kit, especially by bonding, welding or other click-fastening.

According to a first embodiment, the two containers are independent of each other. Such containers may be in various forms. They may especially be tubes, flasks or bottles.

One and/or the other of the containers may have mounted on it a manually actuated pump on which is mounted a push-button for actuating the pump and dispensing the composition via at least one dispensing orifice.

Alternatively, one and/or the other of the containers is pressurized, especially by means of a propellant, in particular a propellent gas. In this case, the container(s) is (are) equipped with a valve on which is mounted a push-button equipped with a nozzle or any other diffusion means for dispensing the product.

The propellant may be admixed with the composition to be dispensed or separated, especially via a piston capable of sliding inside the container, or via the flexible walls of a pocket inside which is placed the composition.

The containers may be made of various materials: plastic, glass or metal.

According to one preferred embodiment, the two compositions are contained inside a single device.

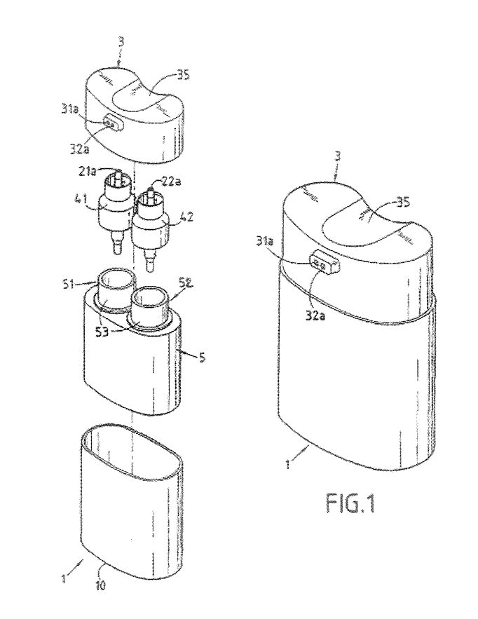

According to the embodiment shown in FIG. 1, the packaging device has two compartments 51, 52 arranged side by side and formed inside a component 5 obtained by moulding a thermoplastic material. Each of the containers 51, 52 comprises a neck 53 delimiting an opening. Inside the neck of each of the containers is mounted a pump 41, 42, which may be with or without air intake.

During mounting, the component 5 delimiting the two compartments 51, 52 is arranged inside a housing element 10.

A pump stalk 21a, 22a, for each of the pumps 41, 42 is forced-fitted inside a corresponding channel provided in a single push-button 3 configured so as to allow the simultaneous actuation of the two pumps, in response to a pressure exerted axially on a surface 35 of the push-button 3.

The push-button channels connected to each of the pumps emerge at two orifices 31a, 32a, arranged close to each other on an outer surface of the push-button 3. In response to actuation of the pumps 41, 42, the two compositions come out separately, either onto the user's finger, or onto an application pad or cotton wool. Mixing of the two compositions then takes place during the application to the surface to be treated.

According to another embodiment, not shown, the two compartments are pressurized and equipped with a push valve or a rocker valve. The two valves may preferably be actuated by means of the same push-button of the type described in reference to the pump embodiment.

Alternatively also, the two compartments are formed from two concentric compartments formed inside a tube, and mounted on them is a pump without air intake, equipped with a push-button comprising one or two dispensing orifices. Inside the tube is a piston that rises in the direction of the pump as and when the compositions are withdrawn from the containers. Such dispensing modes are used especially for dispensing toothpastes.

Numerous other devices may be used for the implementation of the present invention, an important feature being that they allow the two compositions to be packaged separately and to be dispensed separately or as a mixture.

As a further example, the two compositions are packaged in two compartments formed inside the same flexible sachet, the two compartments being separated by a rupture zone that can be broken at the time of use, especially in response to a pressure exerted at a precise point on the sachet.

The invention thus relates in particular to a kit, in which compositions A and B are packaged in two compartments formed from two separate containers.

According to one particular mode, compositions A and B are packaged in two compartments (51, 52) delimited by a single device (1) as shown in FIG. 1.

In particular, each of the compartments is equipped with a pump (41, 42), which is preferably manually actuated, connected to at least one actuating and dispensing means (3) for dispensing compositions A and B, separately or as a mixture.

According to one preferred mode, the actuating and dispensing means (3) is common to the two pumps.

According to one alternative, each of the compartments is pressurized, especially by means of a propellant, and equipped with a valve connected to at least one actuating and dispensing means for dispensing compositions A and B, separately or as a mixture.

In particular, the actuating and dispensing means is common to the two valves.

The invention also relates to a skincare and/or scalp-care process comprising the application to the skin or the scalp of a composition A and of a composition B as defined above, either simultaneously, or successively or sequentially over time.

The application will preferably be performed simultaneously.

According to one particular mode, the process according to the invention is intended for caring for greasy skin.

According to another mode, the process according to the invention is intended for caring for dry skin, sensitive skin, delicate skin (e.g.: baby skin) and/or attacked skin (e.g.: shaving, acne treatment, etc.).

The invention will now be described with reference to the examples that follow, which are given as non-limiting illustrations. In these examples, unless otherwise indicated, the amounts are expressed as weight percentages.

### EXAMPLE

### Astringent and Moisturizing Twofold Care

Astringent Composition A at pH 3.5 (Acidic) Containing Alum Crystals

| | |
|---|---|
| Potassium alum | 1.5% |
| Ammonium polyacryloyldimethyltaurate | 2.0% |
| (Hostacerin AMPS ® from Clariant) | |
| Volatile silicones | 6.0% |
| Solvents | 4.0% |
| Stearic acid | 1.0% |
| Emulsifiers | 3.0% |
| Preserving agents | 0.4% |
| Water | qs 100% |

Moisturizing Composition B at pH 7 (Basic) Containing Hydroxyalkyl Urea

| | |
|---|---|
| N-(2-Hydroxyethyl)urea | 5.0% |
| Ammonium polyacryloyldimethyltaurate | 0.5% |
| (Hostacerin AMPS ® from Clariant) | |
| Volatile silicone | 5.0% |
| Silicone polymer (NLK506 ® from | 3.0% |
| Takemoto) | |
| Glycerol | 3.0% |
| Emulsifiers | 1.5% |
| Preserving agents | 0.2% |
| Water | qs 100% |

Compositions A and B are prepared and are then packaged separately in a device as described previously and shown in FIG. 1. A pressure exerted axially on a single push-button 3 simultaneously actuates the two pumps, and allows the two compositions A and B to come out separately onto the user's finger, or onto an application pad or cotton wool. Mixing of the two compositions then takes place during the application to the surface to be treated.

A good astringent and moisturizing effect is obtained after application to the skin.

Test Showing the pH Instability in the Presence of N-(2-hydroxyethyl)urea:

1) Composition of pH 6.4 containing 5% of N-(2-hydroxyethyl)urea

| | |
|---|---|
| N-(2-Hydroxyethyl)urea | 5% |
| Caffeine | 3% |
| Triethanolamine | 0.6% |
| Escin | 0.2% |
| Extract of Ginkgo biloba | 0.05% |
| Extract of butcher's-broom | 0.1% |
| Silica | 0.5% |
| Salicylic acid | 0.5% |
| Ammonium polyacryloyldimethyltaurate | 1.75% |
| (Hostacerin AMPS ® from Clariant) | |
| Volatile silicone | 10% |
| Glycerol | 3% |
| Emulsifiers | 3% |
| Propylene glycol | 6% |
| Ethanol | 15% |
| Water | qs 100% |

The pH at time zero is 6.4, and rises to 6.9 within two weeks, which shows the pH instability of a composition of acidic pH (pH less than 7) containing N-(2-hydroxyethyl)urea.

2) Composition Containing an Increasing Amount of N-(2-hydroxyethyl)urea

| | |
|---|---|
| N-(2-Hydroxyethyl)urea | x% |
| Tetrasodium EDTA | 0.1% |
| Cocoa extract | 1% |
| Oils | 18.5% |
| Sodium acrylate/sodium acryloyldimethyltaurate | 1% |
| copolymer/isohexadecane/Polysorbate | |
| 80 (Simulgel EG from SEPPIC) | |
| Sodium hyaluronate | 0.02% |
| Carbomer | 0.5% |
| Triethanolamine | 0.25% |
| Divinyl dimethicone/dimethicone copolymer/ | 0.5% |
| C12-13 Pareth-23/C12-13 Pareth-3 (HMW2220 | |
| from Dow Corning) | |
| Glycerol | 10% |
| Emulsifiers | 2.5% |
| Ethanol | 3.5% |
| Preserving agents | 0.75% |
| Water | qs 100% |

It is found that the variation of pH after two weeks is greater when the percentage of Hydrovance is greater.

Moreover, after two weeks at 45° C., the composition containing 20% of Hydrovance rises from pH 5.3 to 6.2. High pH instability is thus observed in the presence of Hydrovance.

The above written description of the invention provides a manner and process of making and using it such that any person skilled in this art is enabled to make and use the same, this enablement being provided in particular for the subject matter of the appended claims, which make up a part of the original description and including a skincare and/or scalp-care kit comprising:
a composition A containing a physiologically acceptable acidic medium and at least one skincare and/or scalp-care agent;
a composition B, packaged separately from composition A, composition B containing a physiologically acceptable basic medium and at least one hydroxyalkyl urea of formula (I) 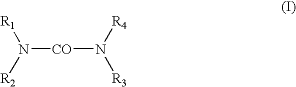
in which R₁, R₂, R₃ and R₄ each independently represent a hydrogen atom, a C₁-C₄ alkyl group or a C₂-C₆ hydroxyalkyl group possibly containing from 1 to 5 hydroxyl groups, at least one of the radicals R₁-R₄ representing a hydroxyalkyl group, and also the salts, solvates and isomers thereof.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. The term “especially” means “preferably.”

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A kit comprising:
a composition A comprising a physiologically acceptable acidic medium and at least one skincare and/or scalp-care agent;
a composition B, packaged separately from composition A, composition B comprising a physiologically acceptable basic medium and at least one hydroxyalkyl urea of formula (I) 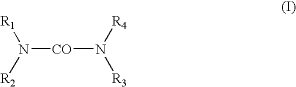
in which R₁, R₂, R₃ and R₄ each independently represent a hydrogen atom, a C₁-C₄ alkyl group or a C₂-C₆ hydroxyalkyl group optionally containing from 1 to 5 hydroxyl groups, at least one of the radicals R₁-R₄ representing a hydroxyalkyl group, and the salt and solvates thereof.

2. The kit according to claim 1, wherein none of the kit, Composition A and Composition B comprise a self-tanning agent or an enzyme.

3. The kit according to claim 1, wherein at least one skincare agent is present in composition A in a content ranging from 0.0001% to 90% by weight relative to the total weight of composition A.

4. The kit according to claim 1, wherein the pH of the acidic medium is less than or equal to 5.

5. The kit according to claim 1, wherein R₁ is a hydroxyalkyl group and R₂, R₃ and R₄ represent, independently of each other, a hydrogen atom or a C₁-C₄ alkyl group.

6. The kit according to claim 5, wherein R₁ is a hydroxyalkyl group and R₂, R₃ and R₄ each represent a hydrogen atom.

7. The kit according to claim 1, wherein composition B comprises at least one compound of formula (I) chosen from N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-[tris(hydroxymethyl)methyl]urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxy-ethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)-urea; and N′,N′-bis(2-hydroxyethyl)-N′,N′-bis(2-hydroxypropyl)urea.

8. The kit according to claim 1, wherein composition B comprises N-(2-hydroxyethyl)urea.

9. The kit according to claim 1, wherein the at least one hydroxyalkyl urea is present in composition B in a content ranging from 0.01% to 50% by weight relative to the total weight of composition B.

10. The kit according to claim 1, wherein composition A comprises at least one care agent chosen from an organic acid, a depigmenting agent, a desquamating agent, an antimicrobial agent, a calmative and/or anti-inflammatory agent, a sebo-regulating agent, a cicatrizing agent, an astringent, a slimming agent, and mixtures thereof.

11. The kit according to claim 1, wherein composition A comprises at least one organic acid chosen from the group consisting of α-hydroxy acids, β-hydroxy acids, α-keto acids, β-keto acids, and mixtures thereof.

12. The kit according to claim 1, wherein composition A comprises at least one organic acid chosen from the group consisting of ascorbic acid, kojic acid, caffeic acid, salicylic acid, citric acid, lactic acid, methyllacetic acid, glucuronic acid, glycolic acid, pyruvic acid, 2-hydroxybutanoic acid, 2-hydroxy-pentanoic acid, 2-hydroxyhexanoic acid, 2-hydroxy-heptanoic acid, 2-hydroxyoctanoic acid, 2-hydroxy-nonanoic acid, 2-hydroxydecanoic acid, 2-hydroxy-undecanoic acid, 2-hydroxydodecanoic acid, 2-hydroxy-tetradecanoic acid, 2-hydroxyhexadecanoic acid, 2-hydroxyoctadecanoic acid, 2-hydroxytetracosanoic acid, 2-hydroxyeicosanoic acid, mandelic acid, benzoic acid, phenylacetic acid, gluconic acid, galacturonic acid, aleuritic acid, ribonic acid, tartronic acid, tartaric acid, malic acid, fumaric acid, retinoic acid, benzene-1,4-bis(3-methylidene-10-camphorsulfonic acid), urocanic acid, 2-phenylbenzimidazole-5-sulfonic acid, α-(2-oxo-3-bornylidene)-4-toluenesulfonic acid, 2-hydroxy-4-methoxy-5-sulfonic acid, plant extracts containing acids, β-glycyrrhetinic acid and asiatic acid, and mixtures thereof.

13. The kit according to claim 1, wherein composition A comprises at least one care agent chosen from ascorbic acid, kojic acid, zinc salts, copper salts, 5-n-octanoylsalicylic acid, (N-2-hydroxyethylpiperazin-N-2-ethane)sulfonic acid, glycolic acid, citric acid, lactic acid, tartaric acid, malic acid or mandelic acid, alum salts, an extract of Hamamelis, an extract of rose, an extract of mint, β-glycyrrhetinic acid, allantoin, serine, arginine, hydroxyproline, caffeine, and nicotinic acid.

14. The kit according to claim 1, wherein composition A comprises at least one care agent present in the physiologically acceptable acidic medium in a content ranging from 0.001% to 90% by weight relative to the total weight of composition A.

15. The kit according to claim 1, wherein composition B further comprises at least one additional skincare and/or scalp-care agent.

16. The kit according to claim 1, wherein compositions A and B are packaged in two compartments formed from two separate containers.

17. The kit according to claim 1, wherein compositions A and B are packaged in two compartments delimited by a single device.

18. The kit according to claim 17, wherein each of the compartments is equipped with a pump connected to at least one actuator and dispenser capable of dispensing compositions A and B separately or as a mixture.

19. The kit according to claim 18, wherein the at least one actuator and dispenser is common to the two pumps.

20. The kit according to claim 17, wherein each of the compartments is pressurized and equipped with a valve connected to at least one actuator and dispenser capable of dispensing compositions A and B separately or as a mixture.

21. The kit according to claim 20, wherein the actuator and dispenser capable of dispensing compositions A and B separately or as a mixture is common to the two valves.

22. A process, comprising applying to the skin:
a composition A comprising a physiologically acceptable acidic medium and at least one skincare and/or scalp-care agent; and
a composition B, comprising a physiologically acceptable basic medium and at least one hydroxyalkyl urea of formula (I) 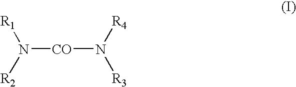
in which R₁, R₂, R₃ and R₄ each independently represent a hydrogen atom, a C₁-C₄ alkyl group or a C₂-C₆ hydroxyalkyl group optionally containing from 1 to 5 hydroxyl groups, at least one of the radicals R₁-R₄ representing a hydroxyalkyl group, and the salt and solvates thereof simultaneously or successively or sequentially over time.

23. The process according to claim 22, wherein said skin is greasy skin, dry skin, sensitive skin, delicate skin and/or attacked skin.
